(19) United States
(12) Patent Application Publication
(10) Pub. No.: US 2026/0355526 A1
(43) Pub. Date: Nov. 12, 2026

(54) COMMUNICATION VIA INDUCTIVE COUPLING

(71) Applicant: Medtronic, Inc., Minneapolis, MN (US)

(72) Inventors: Andrew T. Fried, St. Paul, MN (US); Joel A. Anderson, Brooklyn Park, MN (US); Jacob P. Komarek, Fridley, MN (US); Arthur K. Lai, Minnetonka, MN (US); Kai Anderson-Lantz, Minneapolis, MN (US); Henry Fischer, Cottage Grove, MN (US); Lucas Linnemann, Faribault, MN (US)

(21) Appl. No.: 19/667,699

(22) Filed: May 5, 2026

Related U.S. Application Data
(60) Provisional application No. 63/803,130, filed on May 9, 2025.

(51) Int. Cl. A61N 1/372; G06F 3/01; H04L 25/49; H04L 67/12

(52) U.S. Cl. CPC A61N 1/37223; A61N 1/37247; G06F 3/015; H04L 25/4902; H04L 67/12

(57) ABSTRACT

Systems and techniques are described for transmitting a communication protocol between devices using inductive coupling. In one example, a device includes an inductive coil, a signal generator coupled to the inductive coil, and processing circuitry. The processing circuitry can be configured to receive a command, responsive to receiving the command, generate instructions according to a standard communication protocol, generate, based on the instructions, a plurality of bits representative of the instructions, and control, according to the plurality of bits, the signal generator to output pulse width modulated pulses to the inductive coil that produces a modulated magnetic field.

### CROSS-REFERENCE TO RELATED APPLICATION

This application claims the benefit of and priority to U.S. Provisional Patent Application No. 63/803,130 filed May 9, 2025, the entire disclosure of which is incorporated by reference herein.

### TECHNICAL FIELD

The disclosure relates to device communication, and more specifically to communication using inductive coupling.

### BACKGROUND

Medical devices may be external or implanted and may be used to monitor patient signals such as cardiac activity, biological impedance and to deliver electrical stimulation therapy to patients via various tissue sites to treat a variety of symptoms or conditions such as chronic pain, tremor, Parkinson's disease, diabetes, epilepsy, urinary or fecal incontinence, sexual dysfunction, obesity, or gastroparesis and other conditions. In some examples, the medical devices may communicate with one or more external devices. The communications may include programming the device to operate according to a patient condition as well as transmitting updates on the patient status to, for example, a clinician caring for the patient. A proprietary external programmer may be used to transmit instructions to the medical device via wireless communication protocol. In some examples, medical devices may include a rechargeable electrical power source, or may be powered directly by transmitting energy through tissue. In other examples, a medical device may receive power from a primary cell battery (non-rechargeable) or from line power.

### SUMMARY

In general, the disclosure describes systems, devices, and techniques for converting and transferring digital information according to a standard communication protocol between separate devices using inductive coupling. A transmitting device can utilize pulse width modulation (PWM) of current to a primary inductive coil to encode each bit of a plurality of bits of the information at the standard communication protocol into a modulated magnetic field. The receiving device can receive the modulated magnetic field at a secondary inductive coil and regenerate the digital information by passing the analog signal from the secondary inductive coil through a bandpass filter, an amplifier, and a comparator. In some examples, this technique and hardware can enable off-the-shelf and proprietary devices to communicate with each other via inductive coupling.

In one example, an external device includes an inductive coil; a signal generator coupled to the inductive coil; and processing circuitry configured to: receive a command; responsive to receiving the command, generate instructions according to a standard communication protocol; generate, based on the instructions, a plurality of bits representative of the instructions; and control, according to the plurality of bits, the signal generator to output pulse width modulated pulses to the inductive coil that produces a modulated magnetic field.

In another example, a device includes an inductive coil configured to generate an analog signal from a received modulated magnetic field; a receive circuit configured to generate a digital signal from the analog signal; and processing circuitry configured to: generate, based on the digital signal, a command; and control the device to perform a function according to the command.

In another example, a system includes: an external device comprising: a first inductive coil; a signal generator coupled to the first inductive coil; and first processing circuitry configured to: receive a first command; responsive to receiving the first command, generate instructions according to a standard communication protocol; generate, based on the instructions, a plurality of bits representative of the instructions; and control, according to the plurality of bits, the signal generator to output pulse width modulated pulses to the first inductive coil that produces a modulated magnetic field. The system can also include an implantable medical device comprising: a second inductive coil configured to generate an analog signal from the modulated magnetic field; a receive circuit comprising: a bandpass filter configured to generate a filtered analog signal from the analog signal; a logarithmic amplifier configured to amplify the filtered analog signal to an amplified signal; and a comparator configured to generate a digital signal according to a comparison of the amplified signal to a reference voltage; and second processing circuitry configured to: generate, based on the digital signal, a second command; and control the implantable medical device to perform a function according to the second command.

The details of one or more examples of the disclosure are set forth in the accompanying drawings and the description below. Other features, objects, and advantages of the disclosure will be apparent from the description and drawings, and from the claims.

### BRIEF DESCRIPTION OF DRAWINGS

FIG. 1 is a conceptual diagram illustrating an example medical system of this disclosure that includes an implantable medical device located near an ankle of a patient.

FIG. 2 is a conceptual diagram illustrating an medical system of this disclosure that includes an implantable medical device located near a pelvis of a patient.

FIG. 3 is a block diagram illustrating example components of an example medical device.

FIG. 4 is a block diagram illustrating example components of an example external device.

FIG. 5 is a timing diagram illustrating example elements of transmitting data inductively.

FIG. 6 is a block diagram of an example system for transmitting data via inductive coupling.

FIG. 7 is a sequence diagram of an example sequence for transmitting and receiving information via inductive coupling.

FIG. 8 is a flow chart illustrating an example technique for transmitting information via inductive coupling.

FIG. 9 is a flow chart illustrating an example technique for receiving information and converting the information to digital information via inductive coupling.

### DETAILED DESCRIPTION

The disclosure describes systems, devices, and techniques for facilitating communication between external devices (e.g. a clinical programmer, a client programmer, an onsite server, or an offsite server, a recharger, a wireless recharging device, a patient programmer, clinician programmer, or an application on a patient programmer) and medical devices such as a wearable or implantable medical device. These external devices may include any external devices configured to be involved with the operation of a medical device. Generally, an external device may include any device that can send or receive a signal or information directly or indirectly to or from the medical device. An external device may include a recharger, a programmer, or even computing device (e.g., tablet computer or hand-held computer) that communicates with the medical device through another device. In some examples, one of the external devices may be referred to as an external instrument when it is configured to interact directly with the medical device (e.g., a recharging device, a programmer, or a wireless sensor). Direct interaction may include directly sending a signal to, or receiving a signal from, the medical device. Cell phones and watches currently use NFC for short range communication with payment systems. However, NFC is challenging to implement in medical devices and may not be able to support large amounts of data. As one example, transmission frequencies for NFC may be outside of frequency ranges that are less attenuated when traveling through human tissue for implanted devices.

Medical devices may be programmed using external programmers (e.g., clinician programmers and patient programmers) and, for rechargeable medical devices, receive charging energy from the external device. For example, an implantable medical device may receive energy wirelessly from an external and wireless charging device via an energy charging modality such as inductive charging. However, the external device may require specific hardware that is configured specifically to communicate with the medical device. These external devices may be proprietary and/or require relatively expensive hardware that can generate signals detectable by the medical device.

Indeed, typical cell phones are not able to communicate at short range using inductive technology with implantable medical devices. Inductive telemetry in implantable medical devices typically requires expensive, proprietary transceiver modules and custom framing logic. This situation can increase the cost of programming and using medical devices, reduce access to medical devices, and require patients and clinicians to carry additional programming devices in order to communicate with medical devices.

However, as described herein, external devices may be configured to communicate with implantable medical devices or other devices using standard communication protocols via inductive coupling. For example, a transmitting device can encode digital commands into pulse width modulated inductive signals that generate a modulated magnetic field, and a receiving device can receive the modulated magnetic field and generate an analog signal that is converted back to the digital command for use in controlling the receiving device. In some examples, commercial off-the-shelf components and standardized interfaces (e.g., hardware and/or communication protocols) can be configured as described herein to communicate inductively between various devices, such as between a cell phone or watch and implantable medical devices or any other device at relatively short range. The techniques and hardware described herein can replace typical complexity with a standard interface, such as a standard microcontroller peripheral (UART) plus minimal analog front-end hardware.

The communication described herein can provide UART over inductive communication via PWM on-off keying (OOK) that enables direct cell phone to IMD short range telemetry. In one example, to transmit information, a device may include a general-purpose timer that generates PWM pulses on an inductive coil. Each UART bit (1 start+8 data+1 stop) is represented by enabling or disabling PWM at the timer interrupt rate. Bit duration can be computed at run-time from the timer clock and a selected baud rate (e.g., 2400 baud→416 μs per bit). After the entire frame, the modulation timer and PWM output can be automatically disabled, restoring any shared ADC or radio resources for the device.

In one example, to receive the information inductively, a device can include receive circuitry that may include a simple analog front end (e.g., a band-pass filter and a logarithmic amplifier) that is configured to recover the envelope of the inductive coil. The envelope is sampled via ADC in DMA mode, and an exponential moving average (EMA) computes the RMS envelope and tracks its decay. A dynamic threshold adapts to signal attenuation over distance and coil misalignment. A high-speed comparator can then produce a clean digital waveform, directly feeding any UART-compatible RX pin. This approach can yield a low-cost, interoperable inductive link, without any special transceiver IC or custom bit-banging, and can provide seamless integration with existing UART drivers.

This technique may be used with other interfaces or communication protocols different than UART. Inductive communication between an external human interface device (e.g., a “Mini Remote”) and an implantable medical device can be provided. These techniques can leverage standard components, such as UART peripherals, plus a minimal analog envelope detector, which can reduce dependency on specialized hardware for communication between devices. This technique can provide a new standard for short range communication with payment systems, authentication systems, and implantable devices.

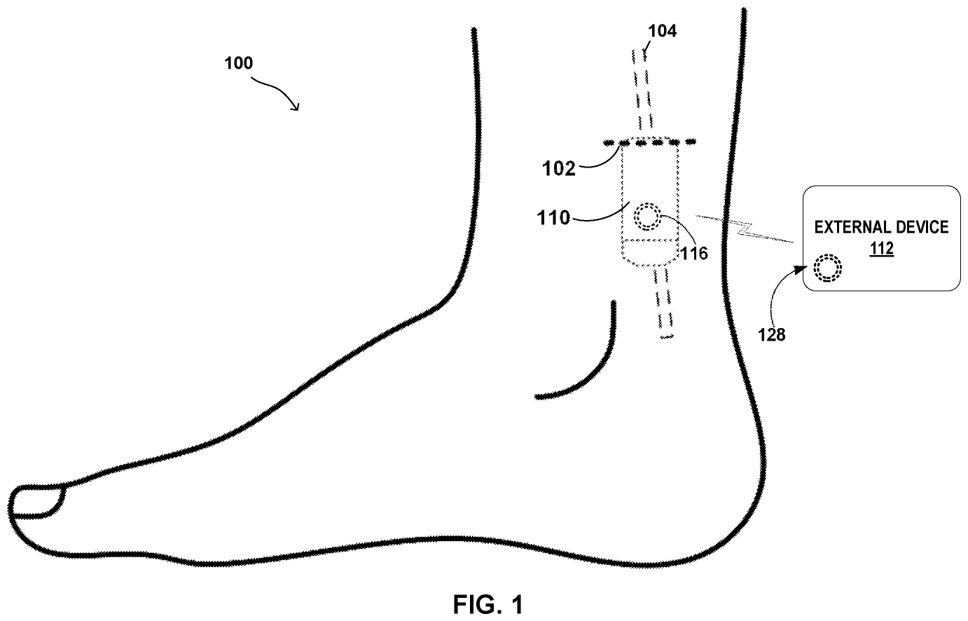

FIG. 1 is a conceptual diagram illustrating an example medical system 100 of this disclosure that includes an implantable medical device located near an ankle of a patient. The example of system 100 in FIG. 1 includes implantable medical device (IMD) 110 and one or more external devices 112.

In some examples, external device 112 may also be referred to as a programmer as shown in the example of FIG. 1, but may be or include a wireless recharger communicator, programmer, patient programmer, clinician programmer, external recharging device, or external computing device in other examples. These examples of external device 112 are all external devices that may be configured to interact directly with IMD 110 (e.g., send signals to or receive signals from a medical device). External device 112 as shown in FIG. 1 includes one or more coils, such as internal transfer coil 128. External device 112 may be used to program or adjust settings of IMD 110 and may also recharge an electrical energy storage device, such as a battery, of IMD 110 (not shown in FIG. 1). In other examples, external device 112 may also be or include a mobile phone, tablet computer, a wearable computing device or similar computing device that includes processing circuitry configured to execute programming instructions to communicate with IMD 110. Such a computing device may communicate with IMD 110 to adjust therapy and/or sensing parameters, download recorded data, and the other functions described in this disclosure. External devices 112 may be configured to send or receive information to any other external devices or IMD 110.

IMD 110 may include sensing circuitry configured to detect biological signals from the patient; electrical stimulation circuitry configured to deliver electrical stimulation to target tissue of the patient, communication circuitry, processing circuitry configured to control the operation of circuitry of IMD 110 and other circuitry (not shown in FIG. 1) configured to perform the functions described in this disclosure. IMD 110 may output the sensed data via communication circuitry (not shown in FIG. 1). In this disclosure, IMD 110 may referred to as medical device 110, implantable medical device (IMD) 110 or, in the example of a neurostimulation medical device, may be referred to as implantable neuro stimulator (INS) 110.

The communication circuitry may be configured to communicate with rechargers in system 100. In some examples, the communication circuitry may establish a first communication channel with external device 112. In some examples, the communication circuitry may also establish a second communication channel with IMD 110. Communication between coil 128 and 116 may utilize inductive coupling where one coil acts as a primary coil that generates a magnetic field and a secondary coil that generates an analog signal as a result of receiving the magnetic field at the secondary coil.

In the example of a rechargeable power source, the rechargeable power source of IMD 110 may include one or more capacitors, batteries, or other components, e.g., chemical, or electrical energy storage devices (not shown in FIG. 1). Example batteries may include lithium-based batteries, nickel metal-hydride batteries, or other materials. The rechargeable power source may be replenished, refilled, or otherwise capable of increasing the amount of energy stored after energy has been depleted. The energy received from transfer coil 116 may be conditioned and/or transformed by a charging circuit. The charging circuit may then send an electrical signal used to charge the rechargeable power source when the power source is fully depleted or only partially depleted.

When the primary coils (e.g., internal transfer coil 128) and the secondary coils (e.g., transfer coil 116) are aligned, or partially aligned, the electrical current in the primary coils may magnetically induce an electrical current in transfer coil 116 within IMD 110. Since the secondary coils are associated with and electrically coupled to a rechargeable power source and/or receive circuitry, the induced electrical current may be used to increase the voltage, or charge level, of the rechargeable power source or transmit information, such as a command or data as described herein.

External device 112 may include a user interface to receive control inputs from a user, such as the patient, medical professional, or other caregiver. External device 112, and any computing device of system 100, may include a touch-screen user interface. The user interface of external device 112 may also provide information to a user, including whether IMD 110 is ON and delivering therapy, whether external device 112 is wirelessly communicating with IMD 110 and other information which external device 112 may transfer or receive. In some examples, external device 112 may have a single user interface that only includes one or two physical buttons instead of a display or touchscreen.

The example of FIG. 1 is a side view of a patient's leg showing IMD 110 as a leadless neurostimulation device near the ankle adjacent to tibial nerve 104. IMD 110 may be implanted through the patient's skin and cutaneous fat layer via incision 102. In some examples, incision 102 may be relatively small (e.g., about one to three cm) to improve patient outcomes and decrease healing time. In some examples, incision 102 may above tibial nerve 104 on a medial aspect of the patient's ankle. While incision 102 is shown approximately horizontal to the length of tibial nerve 104, other incisions 102 or implantation techniques could be used according to physician preference such as a longitudinal to the length of tibial nerve 104 or at a 45 degree angle relative to tibial nerve 104. The example of FIG. 1 describes IMD 110 for tibial nerve stimulation. In other examples, the techniques of this disclosure may apply to other rechargeable devices, such as implantable neurostimulation system for use in spinal cord stimulation therapy, deep brain stimulation, as well as to other types of medical devices without limitation.

IMD 110 may be positioned adjacent to the region defined by flexor digitorum longus and soleus in which tibial nerve 104 is contained and implanted adjacent and proximal to a fascia layer. One or more electrodes of IMD 110 may face toward tibial nerve 104. Though not shown in FIG. 1, IMD 110 may also connect to one or more leads comprising one or more electrodes (not shown in FIG. 1).

IMD 110 may be constructed of any polymer, metal, or composite material sufficient to house the components of IMD 110. In this example, IMD 110 may be constructed with a biocompatible housing, such as titanium or stainless steel, or a polymeric material such as silicone or polyurethane, and surgically implanted at a site in patient near tibial nerve 104, in some examples, while in other examples, implanted near the pelvis, abdomen, or buttocks. The housing of IMD 110 may be configured to provide a hermetic seal for components, such as a rechargeable power source. In addition, the housing of IMD 110 may be selected of a material that facilitates receiving energy to charge the rechargeable power source.

During normal operation after implantation, an electrical stimulation signal may be transmitted between one or more electrodes through the fascia layer. The electrical signal may be used to stimulate tibial nerve 104 which may be useful in the treatment of overactive bladder (OAB) symptoms of urinary urgency, urinary frequency and/or urge incontinence, or fecal incontinence.

One type of therapy for treating bladder dysfunction includes delivery of electrical stimulation to a target tissue site within a patient to cause a therapeutic effect during delivery of the electrical stimulation. For example, delivery of electrical stimulation from IMD 110 to a target therapy site, e.g., a tissue site that delivers stimulation to modulate activity of tibial nerve 104, spinal nerve (e.g., a sacral nerve), a pudendal nerve, dorsal genital nerve, an inferior rectal nerve, a perineal nerve, or branches of any of the aforementioned nerves, may provide a therapeutic effect for bladder dysfunction, such as a reduction in frequency of bladder contractions. In some cases, electrical stimulation of tibial nerve 104 may modulate afferent nerve activities to restore urinary function.

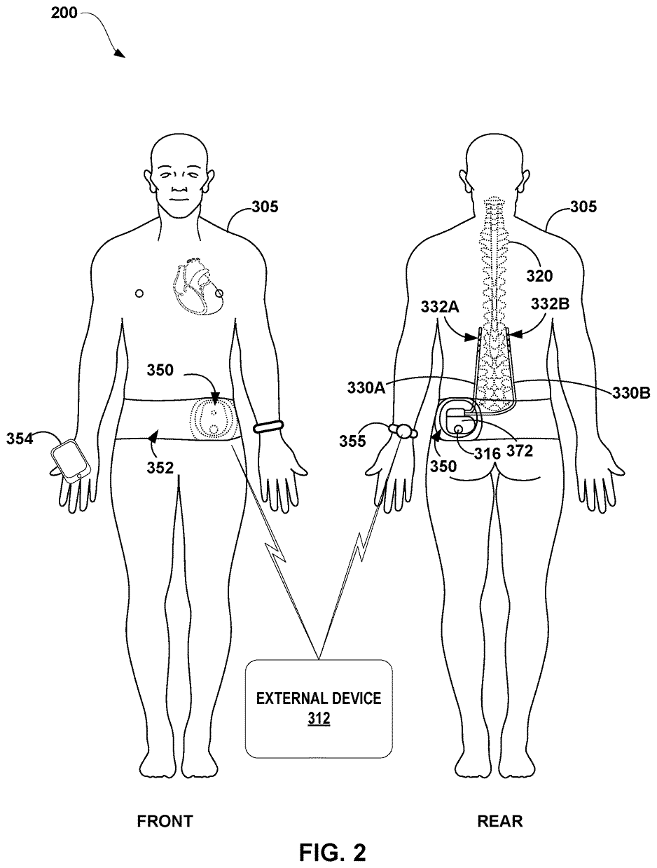

FIG. 2 is a conceptual diagram illustrating a medical system of this disclosure that includes an implantable medical device located near a pelvis of a patient. In the example shown, system 200 includes IMD 372 configured to deliver spinal cord stimulation (SCS) therapy and external instrument 350, in accordance with one or more techniques of this disclosure. Although the techniques described in this disclosure are generally applicable to a variety of medical devices including external devices and IMDs, application of such techniques to IMDs and, more particularly, implantable electrical stimulators (e.g., neurostimulators) will be described for purposes of illustration. More particularly, the disclosure will refer to an implantable SCS system for purposes of illustration, but without limitation as to other types of medical devices or other therapeutic applications of medical devices. In the example of FIG. 2, system 200 includes IMD 372 with antenna 316, external instrument 350, and external device 312, which may be similar to examples of IMD 110 with coil 116, external device 112 described above in relation to FIG. 1 and may have the same or similar functions and characteristics.

As shown in FIG. 2, system 200 includes an IMD 372, leads 330A and 330B, and external instrument 350 shown in conjunction with a patient 305, who is ordinarily a human patient. In the example of FIG. 2, IMD 372 is an implantable electrical stimulator that is configured to generate and deliver electrical stimulation therapy to patient 305 via one or more electrodes of electrodes 332A and 332B, respectively on leads 330A and/or 330B (collectively, “leads 330”), e.g., for relief of chronic pain or other symptoms. In other examples, IMD 372 may be coupled to a single lead carrying multiple electrodes or more than two leads each carrying multiple electrodes, or leadless, as in the example of IMD 110 depicted in FIG. 1. IMD 372 may include an electrical connector configured to connect to the electrical leads, e.g., in the header of IMD 372.

IMD 372 may be a chronic electrical stimulator that remains implanted within patient 305 for weeks, months, or even years. In other examples, IMD 372 may be a temporary, or trial, stimulator used to screen or evaluate the efficacy of electrical stimulation for chronic therapy. In one example, IMD 372 is implanted within patient 305, while in another example, IMD 372 is an external device coupled to percutaneously implanted leads.

In the example of FIG. 2, external instrument 350 may be placed near IMD 372 to communicate and/or transfer power to IMD 372. In some examples, external instrument 350 may be held in place by a belt or straps 352. In some examples belt 352 may include a pouch that accepts external instrument 350. Any of the computing devices of system 300 may include a user interface. Examples of the user interface may include indicator lights, audio feedback, or graphics displayed on a graphical user interface (GUI) such as a tablet computer, smart phone 354, wearable computing device 355 or similar device.

As described above in relation to FIG. 1, in some examples, a user, such as a clinician or patient 305, may interact with a user interface of external instrument 350 or external device 312, to program IMD 372, download collected patient data, and similar interactions. Communication circuitry of system 200, located on any of IMD 372 and the rechargers of system 300 may establish one or more communication channels on a communication link. External device 312 may inductively couple with IMD 372 to deliver programming commands and/or receive information from IMD 372.

Programming of IMD 372 may refer generally to the generation and transfer of commands, programs, or other information to control the operation of IMD 372. In this manner, IMD 372 may receive the transferred commands and programs from external instrument 350 to control stimulation, such as electrical stimulation therapy (e.g., informed pulses), control stimulation (e.g., control pulses), haptic stimulation, sensing and other operating parameters. When recharging, external instrument 350 may communicate to IMD 372 with a more limited information set than a patient programmer or clinician programmer.

For example, a clinician programmer may transmit therapy stimulation programs, evoked compound action potential (ECAP) test stimulation programs, stimulation parameter adjustments, therapy stimulation program selections, ECAP test program selections, user input, or other information to control the operation of IMD 372 as described above in relation to FIG. 1. A patient programmer device, or wearable computing device 355 and mobile computing device 354 with processing circuitry executing an application configured to control the operation of IMD 372, may communication with a more limited information set and at a different authorization level than a clinical programmer.

As described above in relation to FIG. 1, information may be transmitted between external instruments of system 200 and IMD 372. Therefore, IMD 372 and the external instruments may communicate via wireless communication using any techniques known in the art. Examples of communication techniques may include, for example, radiofrequency (RF) telemetry and inductive coupling, but other techniques are also contemplated. In some examples, external device 112 includes a communication head, e.g., transfer coil 128 depicted in FIG. 1, that may be placed proximate to the patient's body near the IMD 372 implant site to improve the quality or security of communication between IMD 372 and external device 312. Communication between the rechargers of system 200 and IMD 372 may occur during power transmission or separate from power transmission.

In some examples, the stimulation signals, or pulses, may be configured to elicit detectable ECAP signals that IMD 372 may use to determine the posture state occupied by patient 305 and/or determine how to adjust one or more parameters that define stimulation therapy. The processing circuitry of IMD 372 may cause the stimulation signals to also deliver haptic stimulation, e.g., stimulation above a perception threshold of patient 305, to provide patient feedback, such as feedback on the quality of alignment between the primary and secondary coils.

The techniques of this disclosure may also apply to other devices, including wearable devices that may be located elsewhere on patient 305. Some examples of IMD 372 may include devices implanted pectorally for deep brain stimulation (DBS), near the tibial region (e.g. to stimulate a tibial nerve such as IMS 110 of FIG. 1 to stimulate tibial nerve 104 of FIG. 1), near the buttocks or flank for sacral neuromodulation, near the heart for cardiac therapy and/or monitoring, and other locations.

In other words, although in one example IMD 372 takes the form of an SCS device, in other examples, IMD 372 takes the form of any combination of DBS devices, sacral neuromodulation (SNM) devices, implantable cardioverter defibrillators (ICDs), pacemakers, cardiac resynchronization therapy devices (CRT-Ds), left ventricular assist devices (LVADs), implantable sensors, orthopedic devices, or drug pumps, as examples. Moreover, techniques of this disclosure may be used to determine parameters that affect stimulation thresholds (e.g., perception thresholds and detection thresholds) associated any one of the aforementioned IMDs and then use a stimulation threshold to inform the intensity (e.g., stimulation levels) of therapy. For example, changing stimulation parameters such as the number of pulses in a burst, the number of bursts over a duration, the pulse width of a pulse in a burst, the ON-time, the OFF-time, a pattern of pulses over a duration and other parameters may change the intensity as well as the efficacy of the therapy to relieve the symptoms.

As with IMD 110 described above in relation to FIG. 1, IMD 372 may be constructed of any polymer, metal, or composite material sufficient to house the components of IMD 372 within patient 305. In this example, IMD 372 may be constructed with a biocompatible housing, such as titanium or stainless steel, or a polymeric material such as silicone, polyurethane, or a liquid crystal polymer, and surgically implanted at a site in patient 305 near the pelvis, abdomen, or buttocks. In other examples, IMD 372 may be implanted within other suitable sites within patient 305, which may depend, for example, on the target site within patient 305 for the delivery of electrical stimulation therapy. The outer housing of IMD 372 may be configured to provide a hermetic seal for components, such as a rechargeable or non-rechargeable power source. In addition, in some examples, the outer housing of IMD 372 is selected from a material that facilitates receiving energy to charge the rechargeable power source.

IMD 372 may deliver electrical stimulation energy, which may be constant current or constant voltage pulses, for example, to one or more target tissue sites of patient 305 via one or more electrodes 332A and 332B (collectively electrodes 332) of implantable leads 330. In the example of FIG. 2, leads 330 carry electrodes that are placed adjacent to the target tissue of spinal cord 320. One or more of electrodes 332 may be disposed at a distal tip of a lead 330 and/or at other positions at intermediate points along the lead. Leads 330 may be implanted and coupled to IMD 372. Electrodes 332 may transfer electrical stimulation generated by an electrical stimulation generator in IMD 372 to tissue of patient 305. Electrodes 332 may also sense bioelectrical signals of patient 305.

Although leads 330 may each be a single lead, lead 330 may include a lead extension or other segments that may aid in implantation or positioning of lead 330. In some other examples, IMD 372 may be a leadless stimulator with one or more arrays of electrodes arranged on a housing of the stimulator rather than leads that extend from the housing, as shown in IMD 110 of FIG. 1. In addition, in some other examples, system 300 may include one lead or more than two leads, each coupled to IMD 372 and directed to similar or different target tissue sites.

Electrodes 332A and 332B of leads 330 may be electrode pads on a paddle lead, circular (e.g., ring) electrodes surrounding the body of the lead, conformable electrodes, cuff electrodes, segmented electrodes (e.g., electrodes disposed at different circumferential positions around the lead instead of a continuous ring electrode), any combination thereof (e.g., ring electrodes and segmented electrodes) or any other type of electrodes capable of forming unipolar, bipolar or multipolar electrode combinations for therapy. Ring electrodes arranged at different axial positions at the distal ends of lead 330 will be described for purposes of illustration.

The deployment of electrodes 332A and 332B via leads 330 is described for purposes of illustration, but arrays of electrodes may be deployed in different ways. For example, a housing associated with a leadless stimulator may carry arrays of electrodes, e.g., rows and/or columns (or other patterns), to which shifting operations may be applied. Such electrodes may be arranged as surface electrodes, ring electrodes, or protrusions. As a an alternative, electrode arrays may be formed by rows and/or columns of electrodes on one or more paddle leads. In some examples, electrode arrays include electrode segments, which are arranged at respective positions around a periphery of a lead, e.g., arranged in the form of one or more segmented rings around a circumference of a cylindrical lead. In other examples, one or more of leads 330 are linear leads having 8 ring electrodes along the axial length of the lead. In another example, the electrodes are segmented rings arranged in a linear fashion along the axial length of the lead and at the periphery of the lead.

The stimulation parameter set of a therapy stimulation program that defines the stimulation pulses of electrical stimulation therapy by IMD 372 through the electrodes of leads 330 may include information identifying which electrodes have been selected for delivery of stimulation according to a stimulation program, the polarities of the selected electrodes, i.e., the electrode combination for the program, voltage or current amplitude, pulse frequency, pulse width, pulse shape of stimulation delivered by the electrodes. These stimulation parameters values that make up the stimulation parameter set that defines pulses may be predetermined parameter values defined by a user and/or automatically determined by system 300 based on one or more factors or user input.

Similarly, sensing bioelectrical signals may use a variety of combinations of electrodes on leads 330, the housing of IMD 372, or other sensors connected directly or indirectly to IMD 372. In some examples IMD 372 may measure and detect other bioelectrical signals from patient 305 including cardiac activity, thoracic impedance, water retention and other signals. In some examples, lead 330 includes one or more sensors configured to allow IMD 372 to monitor one or more parameters of patient 305, such as patient activity, pressure such as blood pressure, temperature, or other characteristics. The one or more sensors may be provided in addition to, or in place of, therapy delivery by lead 330.

Although FIG. 2 is directed to SCS therapy, e.g., used to treat pain, in other examples system 300 may be configured to treat any other condition that may benefit from electrical stimulation therapy. For example, system 300 may be used to treat tremor, Parkinson's disease, epilepsy, a pelvic floor disorder (e.g., urinary incontinence or other bladder dysfunction, fecal incontinence, pelvic pain, bowel dysfunction, or sexual dysfunction), obesity, gastroparesis, or psychiatric disorders (e.g., depression, mania, obsessive compulsive disorder, anxiety disorders, and the like). In this manner, system 300 may be configured to provide therapy taking the form of deep brain stimulation (DBS), peripheral nerve stimulation (PNS), peripheral nerve field stimulation (PNFS), cortical stimulation (CS), pelvic floor stimulation, gastrointestinal stimulation, or any other stimulation therapy capable of treating a condition of patient 305. In other examples, IMD 372 takes the form of any combination of deep brain stimulation (DBS) devices, implantable cardioverter defibrillators (ICDs), pacemakers, cardiac resynchronization therapy devices (CRT-Ds), left ventricular assist devices (LVADs), implantable sensors, orthopedic devices, drug pumps and so on.

IMD 372 is configured to deliver electrical stimulation therapy to patient 305 via selected combinations of electrodes carried by one or both of leads 330, alone or in combination with an electrode carried by or defined by an outer housing of IMD 372. The target tissue for the electrical stimulation therapy may be any tissue affected by electrical stimulation, which may be in the form of electrical stimulation pulses or continuous waveforms. In some examples, the target tissue includes nerves, smooth muscle, or skeletal muscle. In the example illustrated by FIG. 2, the target tissue is tissue proximate spinal cord 320, such as within an intrathecal space or epidural space of spinal cord 320, or, in some examples, adjacent nerves that branch off spinal cord 320. Leads 330 may be introduced into spinal cord 320 in via any suitable region, such as the thoracic, cervical, or lumbar regions. Stimulation of spinal cord 320 may, for example, reduce or limit pain signals from traveling through spinal cord 320 and to the brain of patient 305. Patient 305 may perceive the interruption of pain signals as a reduction in pain and, therefore, efficacious therapy results. In other examples, stimulation of spinal cord 320 may produce paresthesia which may be reduce the perception of pain by patient 305, and thus, provide efficacious therapy results.

IMD 372 is configured to generate and deliver electrical stimulation therapy to a target stimulation site within patient 305 via the electrodes of leads 330 to patient 305 according to one or more therapy stimulation programs. A therapy stimulation program defines values for one or more parameters (e.g., a parameter set) that define an aspect of the therapy delivered by IMD 372 according to that program. For example, a therapy stimulation program that controls delivery of stimulation by IMD 372 in the form of pulses may define values for voltage or current pulse amplitude, pulse width, pulse rate (e.g., pulse frequency), electrode combination, pulse shape, etc. for stimulation pulses delivered by IMD 372 according to that program. In some examples, parameters may include sequences of pulses, for example a “burst” of pulses with gradually increasing current magnitudes, or some other sequence. In some examples, IMD 372 may deliver therapy for a given duration and stop delivering therapy for a given duration. In other words, parameters of the electrical stimulation therapy may include an ON-time and an OFF-time. In some examples, an ON-time may be a few seconds or minutes and the OFF-time may also be for a few seconds or minutes. The ON-time may be equal to the OFF-time in some examples, while in other examples the ON-time and the OFF-time may be unequal durations.

In some examples, IMD 372 may be configured to deliver control stimulation to patient 305 via a combination of electrodes of leads 330, alone or in combination with an electrode carried by or defined by an outer housing of IMD 372 to detect ECAP signals (e.g., control pulses and/or informed pulses). The tissue targeted by the stimulation may be the same or similar tissue targeted by the electrical stimulation therapy, but IMD 372 may deliver stimulation pulses for ECAP signal detection via the same, at least some of the same, or different electrodes. Since control stimulation pulses may be delivered in an interleaved manner with informed pulses (e.g., when the pulses configured to contribute to therapy interfere with the detection of ECAP signals or pulse sweeps intended for posture state detection via ECAP signals do not correspond to pulses intended for therapy purposes), a clinician and/or user may select any suitable electrode combination for informed pulses. Like the electrical stimulation therapy, the control stimulation may be in the form of electrical stimulation pulses or continuous waveforms.

In one example, each control stimulation pulse may include a balanced, bi-phasic square pulse that employs an active recharge phase. However, in other examples, the control stimulation pulses may include a monophasic pulse followed by a passive recharge phase. In other examples, a control pulse may include an imbalanced bi-phasic portion and a passive recharge portion. Although not necessary, a bi-phasic control pulse may include an interphase interval between the positive and negative phase to promote propagation of the nerve impulse in response to the first phase of the bi-phasic pulse. The control stimulation may be delivered without interrupting the delivery of the electrical stimulation informed pulses, such as during the window between consecutive informed pulses. The control pulses may elicit an ECAP signal from the tissue, and IMD 372 may sense the ECAP signal via two or more electrodes on leads 330. In cases where the control stimulation pulses are applied to spinal cord 320, the signal may be sensed by IMD 372 from spinal cord 320.

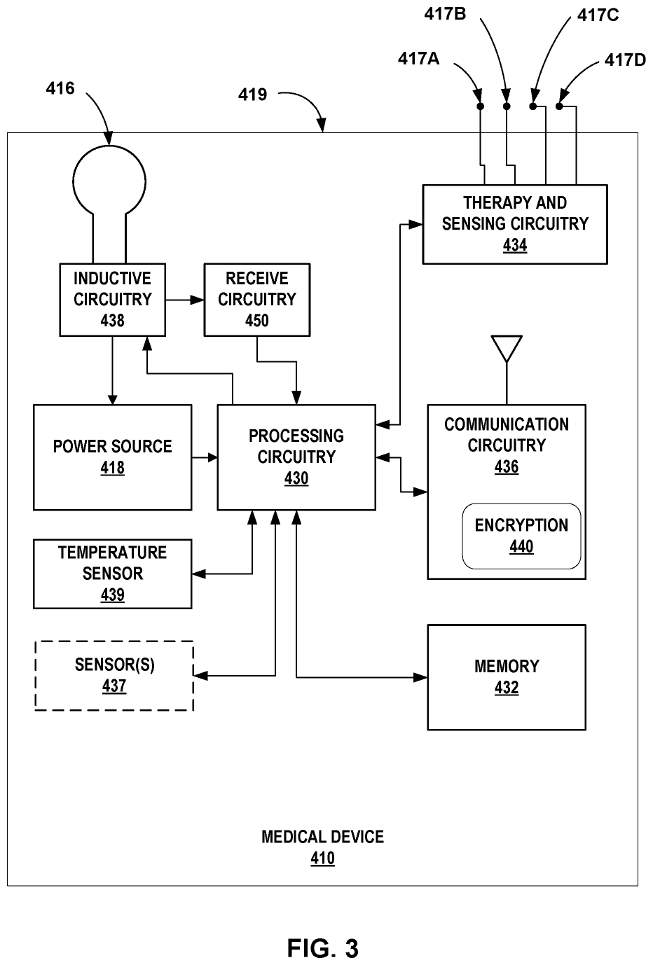

FIG. 3 is a block diagram illustrating example components of the medical device as described above. Implantable medical device (IMD) 410 may be an example of IMD 110 or IMD 372 of FIGS. 1 and 2, respectively. IMD 410 of FIG. 3 is described as an implantable medical device, but the same functions, characteristics and techniques may also apply to other type of wearable or portable medical devices.

In the example illustrated in FIG. 3, IMD 410 includes housing 419 which may contain coil 416, power source 418, processing circuitry 430, memory 432, therapy and sensing circuitry 434, communication circuitry 436, one or more sensors 437, inductive circuitry 438, receive circuitry 450, temperature sensor 439, and encryption/decryption circuitry 440. IMD 410 may be connected to electrodes 417. In other examples, IMD 410 may include a greater or a fewer number of components, e.g., in some examples, IMD 410 may not include sensors 437 or temperature sensor 439. In general, IMD 410 may comprise any suitable arrangement of hardware, alone or in combination with software and/or firmware, to perform the various techniques described herein attributed to IMD 410 and processing circuitry 430, and any equivalents thereof.

Processing circuitry 430 of IMD 410 may be implemented as one or more processors, such as one or more microprocessors, digital signal processors (DSPs), application specific integrated circuits (ASICs), field programmable gate arrays (FPGAs), or any other equivalent integrated or discrete logic circuitry, as well as any combinations of such components. IMD 410 may include a memory 432, such as random access memory (RAM), read only memory (ROM), programmable read only memory (PROM), erasable programmable read only memory (EPROM), electronically erasable programmable read only memory (EEPROM), flash memory, comprising executable instructions for causing the processing circuitry 430 to perform the actions attributed to this circuitry.

Moreover, although processing circuitry 430, therapy and sensing circuitry 434, communication circuitry 436, inductive circuitry 438, receive circuitry 450, and temperature sensor 439 are described as separate modules, in some examples, some combination of processing circuitry 430, therapy and sensing circuitry 434, communication circuitry 436, inductive circuitry 438, receive circuitry 450, and temperature sensor 439 are functionally integrated. In some examples, processing circuitry 430, therapy and sensing circuitry 434, communication circuitry 436, inductive circuitry 438, receive circuitry 450, and temperature sensor 439 correspond to individual hardware units, such as ASICs, DSPs, FPGAs, or other hardware units. For example, components of IMD 410 may be implemented as separate circuits in some examples. In other examples, two or more components of IMD 410 may be implemented on a single integrated circuit, e.g., including processing circuitry 430, receive circuitry 450, memory 432, therapy and sensing circuitry 434, communication circuitry 436, and other components of IMD 410. In this disclosure, therapy, and sensing circuitry 434 may be referred to as therapy circuitry 434.

Memory 432 may store therapy programs or other instructions that specify therapy parameter values for the therapy provided by therapy circuitry 434 and IMD 410. In some examples, memory 432 may also store temperature data from temperature sensor 439, instructions for recharging rechargeable power source 418, thresholds, instructions for communication between IMD 410 and an external instrument (such as external device 112 or 312 of FIGS. 1 and 2, respectively), or any other instructions required to perform tasks attributed to IMD 410. Memory 432 may be configured to store instructions for communication with and/or controlling one or more temperature sensors of temperature sensor 439. In various examples, memory 432 stores information related to determining the temperature of housing 419 and/or exterior surface(s) of housing 419 of IMD 410 based on temperatures sensed by one or more temperature sensors, such as temperature sensor 439, located within IMD 410.

For example, memory 432 may store programming settings such as parameters for electrical stimulation therapy output, e.g., magnitude, pulse width, and so on. Memory 432 may store parameters and other settings for the delivery of haptic stimulation. Settings may be individualized based on patient preference and/or patient physiology. For example, a stimulation intensity that is above the perception threshold for a first patient may be different than the stimulation intensity that may be above the perception threshold for a second patient. In some examples, a patient may find a particular frequency to be annoying or painful and therefore, may request a different frequency setting when receiving haptic stimulation as feedback.

Instructions stored at memory 432 when executed by processing circuitry 430 may determine whether a sensed bioelectrical signal is valid, such as and ECAP or other signal in response to an output electrical stimulation therapy event. Memory 432 may store programming instructions that when executed by processing circuitry 430 cause processing circuitry 430 to cause electrical stimulation circuitry therapy circuitry 434 to deliver electrical stimulation therapy to a target nerve of a patient. Memory 432 may also store instructions on encrypting and decrypting communications to be sent via communications circuitry 436 to an external device (such as external device 112 or 312 of FIGS. 1 and 2, respectively), as well as instructions for establishing a communication channel over communication channels, as described above in relation to FIGS. 1 and 2.

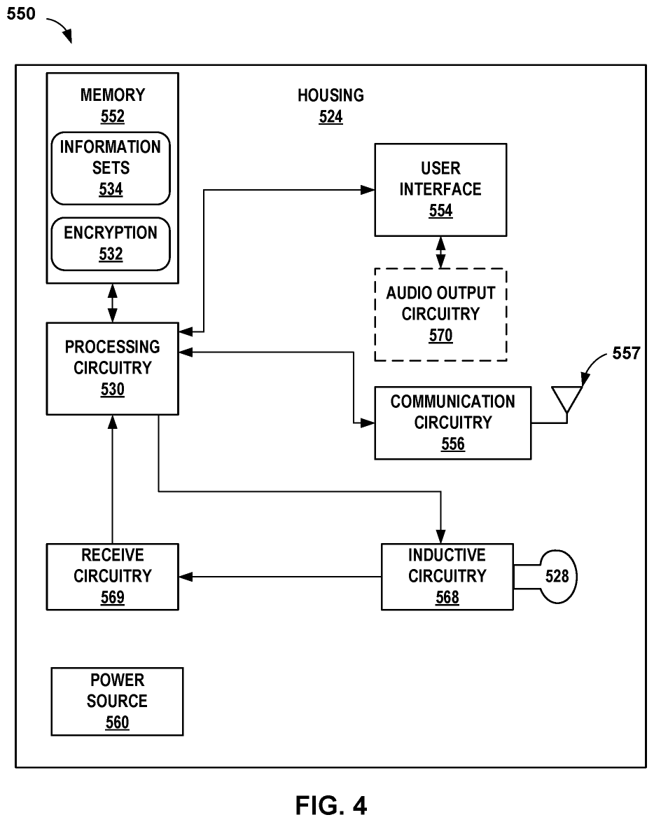

Therapy and sensing circuitry 434 may generate and deliver electrical stimulation under the control of processing circuitry 430. Therapy and sensing circuitry 434 may also output non-therapy stimulation, such as control pulses and haptic stimulation. In some examples, processing circuitry 430 controls therapy circuitry 434 by accessing memory 432 to selectively access and load at least one of the stimulation programs to therapy circuitry 434. For example, in operation, processing circuitry 430 may access memory 432 to load one of the stimulation programs to therapy circuitry 434. In such examples, relevant stimulation parameters may include a voltage amplitude, a current amplitude, a pulse rate, a pulse width, a duty cycle, or the combination of electrodes 417A, 417B, 417C, and 417D (collectively “electrodes 417”) that therapy circuitry 434 may use to deliver the electrical stimulation signal as well as sense biological signals. In other examples, IMD 410 may have more or fewer electrodes than the four shown in the example of FIG. 4. In some examples, electrodes 417 may be part of or attached to a housing of IMD 410, e.g., a leadless electrode. In other examples, one or more of electrodes 417 may be part of a lead implanted in or attached to a patient to sense biological signals and/or deliver electrical stimulation, as described above in relation to FIG. 1.

In some examples, one or more electrodes 417 connected to therapy circuitry 434 may connect to one or more sensing electrodes 417, e.g., attached to housing of IMD 410. In some examples electrodes 417 may be configured to detect an evoked motor response caused by the electrical stimulation therapy event, or other bioelectrical signals such as ECAPs, impedance and so on.

IMD 410 also includes components to receive power to recharge rechargeable power source 418 when rechargeable power source 418 has been at least partially depleted. As shown in FIG. 3, IMD 410 includes coil 416 and inductive circuitry 438 coupled to rechargeable power source 418. Inductive circuitry 438 may be configured to charge rechargeable power source 418 with the selected power level determined by either processing circuitry 430 or an external charging device, such as external computing IMD 110 described above in relation to FIG. 1. Inductive circuitry 438 may include any of a variety of charging and/or control circuitry configured to process or convert current induced in coil 416 into charging current to charge power source 418. For example, inductive circuitry 438 may include measurement circuitry configured to determine a magnitude of current received by secondary coil 416, a magnitude of current delivered to power source 418, and other measurements. Inductive circuitry 438 may send such measurements to processing circuitry 430 to be used in system metrics, and sent to the external instrument (such as external device 112 or 312 of FIGS. 1 and 2, respectively) via communication circuitry 436. Coil 416 may be configured to have a resonant frequency selected from a range of about 9 kHz to about 200 kHz, such as about 175 kHz in one example.

Receive circuitry 450 may include one or more components configured to convert received signals from coil 416 to digital information according to a standard protocol such as UART. Receive circuitry 450 may include a bandpass filter configured to generate a filtered analog signal from the analog signal generated by coil 416 from a magnetic field, a logarithmic amplifier configured to amplify the filtered analog signal to an amplified signal, and a comparator configured to generate a digital signal according to a comparison of the amplified signal to a reference voltage. Processing circuitry 430 can then use the digital signal as a command or instructions to perform various actions for IMD 419.

Secondary coil 416 may include a coil of wire or other device capable of inductive coupling with a primary coil disposed external to the patient. Although secondary coil 416 is illustrated as a simple loop of in FIG. 3, secondary coil 416 may include multiple turns of conductive wire. Secondary coil 416 may include a winding of wire configured such that an electrical current may be induced within secondary coil 416 from a magnetic field. The induced electrical current may then be used to recharge rechargeable power source 418.

Inductive circuitry 438 may include one or more circuits that process, filter, convert and/or transform the electrical signal induced in the secondary coil to an electrical signal capable of recharging rechargeable power source 418. For example, in alternating current induction, inductive circuitry 438 may include a half-wave rectifier circuit and/or a full-wave rectifier circuit configured to convert alternating current from the induction to a direct current for rechargeable power source 418. The full-wave rectifier circuit may be more efficient at converting the induced energy for rechargeable power source 418. However, a half-wave rectifier circuit may be used to store energy in rechargeable power source 418 at a slower rate. In some examples, inductive circuitry 438 may include both a full-wave rectifier circuit and a half-wave rectifier circuit such that inductive circuitry 438 may switch between each circuit to control the charging rate of rechargeable power source 418 and temperature of IMD 410.

Rechargeable power source 418 may include one or more capacitors, batteries, and/or other energy storage devices. Rechargeable power source 418 may deliver operating power to the components of IMD 410. In some examples, rechargeable power source 418 may include a power generation circuit to produce the operating power. Rechargeable power source 418 may be configured to operate through many discharge and recharge cycles. Rechargeable power source 418 may also be configured to provide operational power to IMD 410 during the recharge process. In some examples, rechargeable power source 418 may be constructed with materials to reduce the amount of heat generated during charging. In other examples, IMD 410 may be constructed of materials and/or using structures that may help dissipate generated heat at rechargeable power source 418, inductive circuitry 438, and/or secondary coil 416 over a larger surface area of the housing of IMD 410.

Although rechargeable power source 418, inductive circuitry 438, and secondary coil 416 are shown as contained within the housing of IMD 410, in some implementations, at least one of these components may be disposed outside of the housing. For example, in some implementations, secondary coil 416 may be disposed outside of the housing of IMD 410 to facilitate better coupling between secondary coil 416 and the primary coil of external charging device. In other examples, power source 418 may be a primary power cell and IMD 410 may not include recharge circuitry 438 and secondary coil 416.

Processing circuitry 430 may also control the exchange of information with an external instrument using communication circuitry 436. Processing circuitry 430 may transmit operational information and receive therapy programs or therapy parameter adjustments over an established secure communication channel via communication circuitry 436. Also, in some examples, IMD 410 may communicate with other implanted devices, such as stimulators, control devices, or sensors, via communication circuitry 436. Communication circuitry 436 may include one or more antennas 437 configured to communicate with the external instrument (such as external device 112 and 312 of FIGS. 1 and 2, respectively), e.g., for power transfer or for data transfer with the other devices. In addition, communication circuitry 436 may be configured to control the exchange of information related to sensed and/or determined temperature data, for example temperatures sensed by and/or determined from temperatures sensed using temperature sensor 439. In some examples, communication circuitry 436 may communicate using inductive communication, and in other examples, communication circuitry 436 may communicate using RF frequencies separate from the frequencies used for inductive charging.

In the example of FIG. 3, communication circuitry 436 includes circuitry 440 configured to manage encryption and decryption and may also execute some of the other functions related to establishing communications channels described in in this disclosure. In other examples, the encryption functions of circuitry 440 may be handled by processing circuitry 430, by some other circuitry of IMD 410, or by any combination thereof.

Communication circuitry 436 may be configured to support wireless communication. For example, communication circuitry 436 may be configured to support wireless communication using Bluetooth™ (e.g., BLE and other versions of Bluetooth™, including future versions of Bluetooth™), Wi-Fi™, Near-Field Communication (NFC), Near Field Magnetic Induction (NFMI), Long Term Evolution, 5th generation (LTE/5G), or MedRadio (MICS: Medical Implant Communication Service, MEDS: Medical External Device Service, MBAD: Medical Body Area Network)) between IMD 410 and another computing device, e.g., external devices 112 or 312. In some examples, communication circuitry 436 supports a communication frequency that may correspond to a high frequency or radio frequency, which may be a radio frequency established via Bluetooth, Wi-Fi, Near-Field Communication (NFC), 175 KHz inductive communication, or MICS, for example. Communication circuitry 436 may be configured to receive an inductive sting. Processing circuitry 430 of IMD 410 may receive, as updates to programs (e.g., at least one program parameter), values for various stimulation parameters such as magnitude and electrode combination, from external devices 112 or 312 via communication circuitry 436. In addition, communication circuitry 436 may communicate with an external medical device via proximal inductive interaction of IMD 410, e.g., during recharging. Communication circuitry 436 may send and receive information on a continuous basis, at periodic intervals, or upon request from the recharger. In some examples, communication circuitry 436 may also be referred to as telemetry circuitry in this disclosure.

In some examples, communication circuitry 436 may be configured to receive communications or data via secondary coil 416. This data may be received using the same configuration of secondary coil (e.g., tuned to the same frequency) or include tuning circuitry that can adjust the resonant frequency of secondary coil to receive and/or transmit information to another device, such as to an external instrument using inductive coupling. In other examples, communication circuitry 436 may be coupled to a different coil than secondary coil 416 for receiving and/or transmitting information.

In some examples, processing circuitry 430 may transmit additional information to external charging device related to the operation of rechargeable power source 418, e.g., at a more limited authorization level than for sending and receiving operational programming instructions or parameters. For example, processing circuitry 430 may use communication circuitry 436 to transmit indications that rechargeable power source 418 is completely charged, rechargeable power source 418 is fully discharged, the amount of charging current output by inductive circuitry 438 e.g., to power source 418, or any other charge status of rechargeable power source 418. In some examples, processing circuitry 430 may use communication circuitry 436 to transmit instructions to the external charging device, including instructions regarding control of the charging session, for example instructions to lower the power level or to terminate the charging session, based on the determined temperature of the housing/external surface 419 of the IMD.

Processing circuitry 430 may also transmit information to external charging device that indicates any errors with rechargeable power source 418 that may reduce or limit rechargeable power source 418 from providing operational power to the components of IMD 410. In various examples, processing circuitry 430 may receive, through communication circuitry 436, instructions for algorithms, including formulas and/or values for constants to be used in the formulas that may be used to determine the temperature of the housing 419 and/or exterior surface(s) of housing 419 of IMD 410 based on temperatures sensed by temperature sensor 439 located within IMD 410 during and after a recharging session performed on rechargeable power source 418.

FIG. 4 is a block diagram illustrating example components of the external device of FIGS. 1 and 2. As described above, in some examples, external device 550 may be a hand-held device, while in other examples, external device 550 may be a larger or a non-portable device. In addition, in other examples external device 550 may be included as part of an external programmer or include functionality of an external programmer. As shown in the example of FIG. 4, external device 550 includes two separate components. Housing 524 encloses components such as a processing circuitry 530, memory 552, user interface 554, communication circuitry 556, inductive circuitry 568, coil 523, receive circuitry 569, power source 560, and audio output circuitry 570. In some examples, housing 524 may be coupled to a second housing that includes include inductive circuitry 568, receive circuitry 569, and/or coil 528, which may be an example of coil transfer coil 128 described above in relation to FIG. 1.

Coil 528 may be integrated within housing 524. Coil 528 may be referred to as external transfer coil, which may be substantially equivalent to transfer coil 128 of FIG. 1. Coil 528 may also be referred to as transfer coils, primary transfer coils, energy transfer coils, data transfer coils, or antenna. In some examples, coil 528 may transfer energy, data, or both. In this manner, external device 550 may include one or more coils within housing 524 or include one or more coils outside of the housing 524. Coil 528 may be configured to have a resonant frequency selected from a range of about 9 kHz to about 200 kHz, such as about 175 kHz in one example.

In general, external device 550 comprises any suitable arrangement of hardware, alone or in combination with software and/or firmware, to perform the techniques ascribed to external device 550, and processing circuitry 530, user interface 554, communication circuitry 556, inductive circuitry 568, and receive circuitry 569 of external device 550, and/or any equivalents thereof. In various examples, external device 550 may include one or more processors, such as one or more microprocessors, DSPs, ASICs, FPGAs, or any other equivalent integrated or discrete logic circuitry, as well as any combinations of such components.

Similar to IMD 110 and 410 described above in relation to FIGS. 1 and 3, components of external device 550 shown in FIG. 4 may be implemented as separate circuitry, or combined into one or more integrated circuits. In other words, although processing circuitry 530, communication circuitry 556, inductive circuitry 568, and/or receive circuitry 569 are described as separate modules, in some examples, processing circuitry 530, communication circuitry 556, inductive circuitry 568, and/or receive circuitry 569 are functionally integrated. In some examples, processing circuitry 530, communication circuitry 556, inductive circuitry 568, and/or receive circuitry 569 correspond to individual hardware units, such as ASICs, DSPs, FPGAs, or other hardware units.

External device 550 also, in various examples, may include a memory 552, such as RAM, ROM, PROM, EPROM, EEPROM, flash memory, a hard disk, a CD-ROM, comprising executable instructions for causing the one or more processors to perform the actions attributed to external device 550. Memory 552 may store instructions that, when executed by processing circuitry 530, cause processing circuitry 530 and external device 550 to provide the functionality ascribed to external device 550 throughout this disclosure, and/or any equivalents thereof.

User interface 554 may include buttons, a keypad, indicator lights, a microphone for voice commands, a display, such as a liquid crystal (LCD), light-emitting diode (LED), or cathode ray tube (CRT) and audio output circuitry. In some examples, the display of user interface 554 may be a touch screen. As discussed in this disclosure, processing circuitry 530 may present and receive information relating to the charging of rechargeable power source 418 via user interface 554. For example, user interface 554 may indicate when charging is occurring, quality of the alignment between primary coil 528 or 548 and the secondary coil of the IMD, the selected power level, current charge level of power source 418, duration of the current recharge session, anticipated remaining time of the charging session, sensed temperatures, or any other information. In some examples, processing circuitry 530 may receive some of the information displayed on user interface 554, e.g., via communication circuitry such as communication circuitry 556. In some examples, user interface 554 may provide an indication to the user regarding the quality of alignment between coil 416, depicted in FIG. 3 and coil 548, based on one or more system metrics, such as the charge current to the battery of the IMD.

Processing circuitry 530 may also receive user input via user interface 554. The input may be, for example, in the form of pressing a button on a keypad or selecting an icon from a touch screen. The input may change programmed settings, start, or stop therapy, request starting or stopping a recharge session, a suitable level of charging, or one or more statistics related to charging power source 418 (e.g., the cumulative thermal dose). In this manner, user interface 554 may allow the user to view information related to the operation of IMD 410.

Inductive circuitry 568 may include one or more circuits that generate an electrical current within primary coil 548. Inductive circuitry 568 may generate an alternating current of specified amplitude and frequency in some examples. In other examples, Inductive circuitry 568 may generate a direct current. In any case, inductive circuitry 568 may be capable of generating electrical signals, and subsequent magnetic fields, to transmit various levels of power to IMD 410. In this manner, inductive circuitry 568 may be configured to charge power source 418 of IMD 410 with the selected power level. In some examples, inductive circuitry 568 may convert a standard communication protocol or other digital information and encode that digital information on pulses from coil 528 via pulse width modulation controlled according to the bits of the digital information.

In some examples, external device 550 can also receive information via coil 528. Receive circuitry 450 may include one or more components configured to convert received signals from coil 416 to digital information according to a standard protocol such as UART. Receive circuitry 450 may include a bandpass filter configured to generate a filtered analog signal from the analog signal generated by coil 528 from a magnetic field, a logarithmic amplifier configured to amplify the filtered analog signal to an amplified signal, and a comparator configured to generate a digital signal according to a comparison of the amplified signal to a reference voltage. Processing circuitry 530 can then use the digital signal as a command or instructions to perform various actions for external device 550.

Power source 560 may deliver operating power to the components of external device 550. Power source 560 may also deliver the operating power to drive primary coil 528 during the charging process. Power source 560 may include a battery and a power generation circuit to produce the operating power. In some examples, a battery of power source 560 may be rechargeable to allow extended portable operation. In other examples, power source 560 may draw power from a wired voltage source such as a consumer or commercial power outlet.

Processing circuitry 530 may, when requested, transmit any stored data in memory 552 for review or processing, such as transmitting the stored data to external devices 112 depicted in FIG. 1 via encrypted channel 202 as depicted in FIG. 2. Processing circuitry 530 may be configured to access memory, such as memory 432 of IMD 410 and/or memory 552 of external device 550, to retrieve information comprising instructions, formulas, and determined values for one or more constants. As described above in relation to FIGS. 1-3, processing circuitry 530 and communication circuitry 556 may establish one or more secure communication channels over a communication link. In some examples, processing circuitry 530 may act as a relay to transfer passthrough data via an encrypted channel, such that communication circuitry 556 is incapable of decrypting the information and merely transfers the passthrough data from an external device to an IMD. In some examples, processing circuitry 530 may act as a man-in-the-middle and is able to encrypt and decrypt transferred information during a communication session, and may pass data through communication channels, such that communication circuitry 556 is capable of decrypting the data. In the example of FIG. 4, instructions for establishing the communication channel, handling the encryption handshaking, setting, and resetting the communication session timers, storing encryption keys and other similar functions may be stored at memory 552 (e.g., encryption circuitry 532). In other examples, (not shown in FIGS. 3 and 4) the encryption keys and/or encryption instructions may be stored in a separate encrypted memory in communication with processing circuitry 430 or 530.

Communication circuitry 556 may support wireless communication between IMD 110 of FIG. 1 and external device 550 under the control of processing circuitry 530. Communication circuitry 556 may also be configured to communicate with another computing device via wireless communication techniques, or direct communication through a wired connection. In some examples, communication circuitry 556 may be substantially similar to communication circuitry 436 of IMD 410 of FIG. 3, providing wireless communication via an RF or proximal inductive medium. In some examples, communication circuitry 556 may include an antenna 557, which may take on a variety of forms, such as an internal or external antenna. Although communication circuitry 556 may each include dedicated antennas for communications between these devices, communication circuitry 556, and 436 may instead, or additionally, be configured to utilize inductive coupling from coils 416 and 528 to transfer data.

Examples of local wireless communication techniques that may be employed to facilitate communication between external device 550 and IMD 410 include radio frequency and/or inductive communication according to any of a variety of standard or proprietary communication protocols, or according to other communication protocols such as the IEEE 802.11x or Bluetooth specification sets. In this manner, other external devices may be capable of communicating with external device 550 without needing to establish a secure wireless connection.

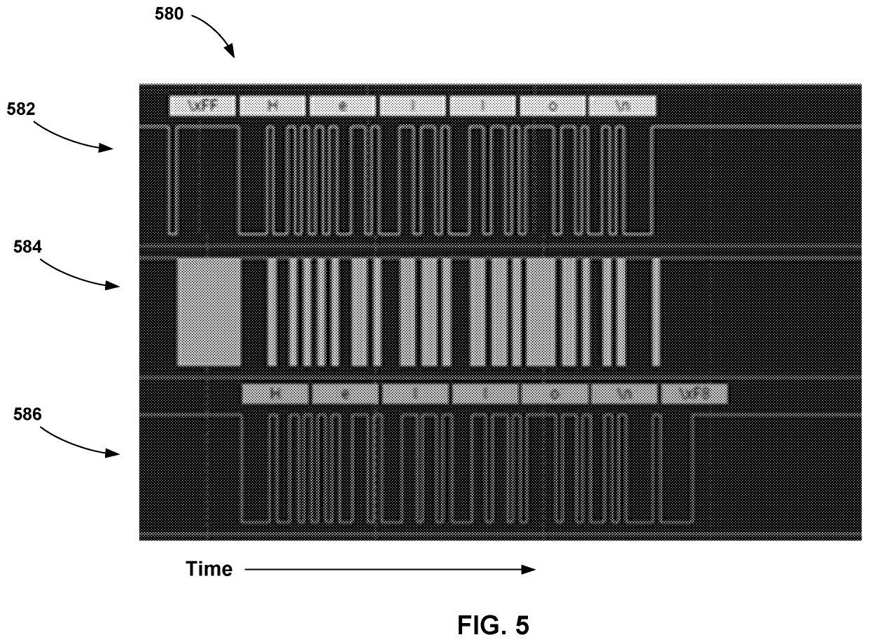

FIGS. 5-7 describe example components and techniques for transmitting and receiving digital commands over inductive coupling. The following processes may be performed by any of the devices described herein.

For example, the transmitting device may utilize timer-driven PWM On-Off Keying (OOK). For implementation, the device may invoke start_bit_modulation(const uint8_t*data, uint16_t size) to initialize and start the timer for the OOK transmission. Within the HAL_TIM_PeriodElapsedCallback (TIM1 ISR), each bit in the pModData buffer is transmitted by calling the following functions: start_pwm( ) for a logical “1” (PWM enabled on TIM3 Channel 1); and stop_pwm( ) for a logical “0” (PWM disabled on TIM3 Channel 1). This alternation produces the on-off keying signal that can be sent to an H-Bridge that drives an inductive coil.

FIG. 5 is a timing diagram illustrating example elements of transmitting data inductively. As shown in FIG. 5, timing diagram 580 includes transmission data 582, transmission PWM modulation 584, and received data 586 via UART. The frame format for the transmission, ma include: 1 start bit (0: PWM off), data bits: 8 data bits LSB-first, and stop bits: 1 stop bit (1: PWM on).

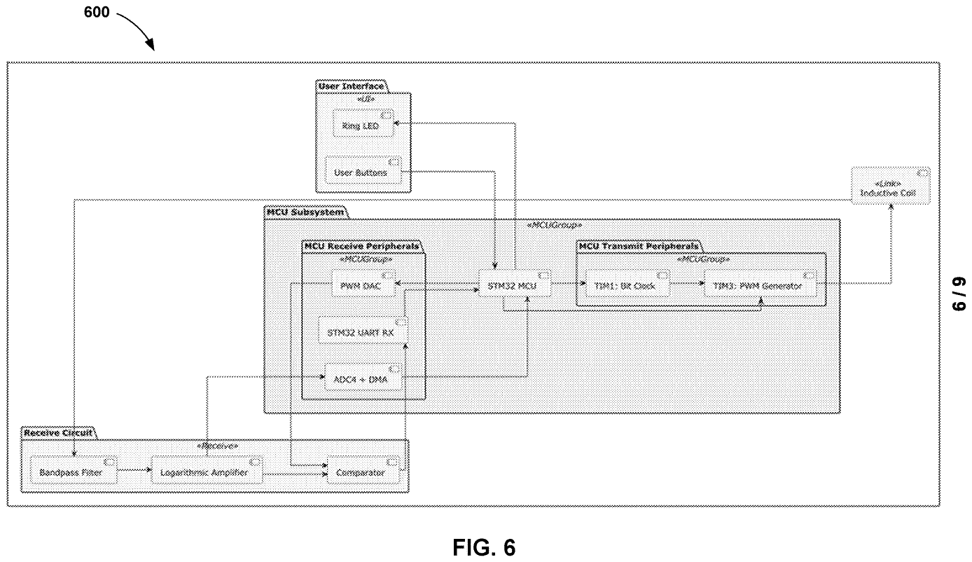

FIG. 6 is a block diagram of an example system 600 for transmitting data via inductive coupling. The components of system 600 are described from the perspective of both transmitting and receiving device. Each transmitting device and receiving device may include all of the components of system 600, but in some examples, only the transmitting device includes transmitting components and receiving device includes receiving components.

The transmitting device may dynamically calculate bit-duration. For example, a component TIM1 fires precisely every bit interval (e.g., 416 μs at 2,400 baud) without blocking loops, letting you support different baud rates by changing BAUD_RATE. The transmitting device can also start sentinel (0xFF) for line framing. The purpose of this can be so that each transmit array begins with 0xFF (all ones→continuous PWM-on). A benefit of this approach may be that the start forces the link high, so the first PWM-off transition unambiguously marks the UART start bit. This sentinel is not payload data. The transmitting device may also use a manual end-of-message newline (‘\n’). For implementation, commands end with ‘\n’ in the array. A benefit of this approach can be that it explicitly signals the receiver to stop collecting bits, preventing noise between messages from being mis-interpreted.

The transmitting device can also provide non-blocking UART trigger & Rx control (e.g., commanding an IMD to change a stimulation parameter value). The process flow can include initiating transmission by calling SendIncrement( ) to begin the sequence. Repeated modulation can be performed by within SendIncrement( ) calling start_bit_modulation(tx_increment, sizeOfInc) three times, with a configured delay between each call. For reception suspension, at the start of each start_bit_modulation( . . . ) invocation, a flag is set to disable UART message processing, preventing the transmitter from interpreting its own output as incoming data. For completion and recovery, once all bits have been sent, TIM1 and the PWM output halt. Clearing the reception-disable flag (Rx_off) restores UART reception.

The receiving device can provide various functions. For envelope sampling with ADC4+DMA, setup includes using enableInductiveTelem( ) to initialize the ADC for inductive communication and starts running it in DMA mode. For a data-ready flag, HAL_ADC_ConvCpltCallback sets adc_data_ready=1 when each 64-sample block finishes. For signal conditioning, a bandpass filter and logarithmic amplifier condition and extract the signal's envelope (RSSI_OUTPUT), which is then fed to a comparator along with a calculated reference voltage to produce a clean, UART-compatible digital waveform (UPLINK_DATA).

Block RMS & Exponential Moving Average (EMA). The device may use the following equation to further process the signal:

$${EMA}_{n} = \{ \begin{matrix}{α}_{attack} * {X}_{n} + ( 1 - {α}_{attack} ) * {EMA}_{n - 1} , & {x}_{n} > {EMA}_{n - 1} \\ {α}_{decay} * {x}_{n} + ( 1 - {α}_{decay} ) * {EMA}_{n - 1} , & {x}_{n} < {EMA}_{n - 1}\end{matrix}$$

To implement, call process_ADC_block( ), abd compute the RMS over the 32 RSSI samples, then update ema_rms using ATTACK_ALPHA and DECAY_ALPHA to smooth spikes and fades.

The system can also provide logarithmic normalization and reference voltage output. This can have the effect of mapping the smoothed envelope into a 0-PWM_MAX duty cycle on TIM3 CH3, using it like a DAC to drive the reference voltage (SLICE_DACOUT) of the comparator.

The receiving device can also provide command parsing and provide a response. The device can receive data using HAL_UART_RxCpltCallback buffers bytes until ‘\n’. For processing, the system can call processReceivedData( ) using containsSubstring( ) to detect “inc”, “dec”, “stop”, “cnt”, or “ble” and then calls the corresponding LED or BLE routines. One of the strengths of using UART is it can be easy to read and understand what the message is trying to convey or what function(s) it should be linked with.

The receiving device can have a flag-driven, low-power architecture. Flags can include volatile flags (adc_data_ready, uart_tx_complete, process_uart, msg_recieved, Rx_off) coordinate main-loop tasks with ISRs. For power saving, after modulation, HAL_TIM_Base_Stop_IT( ) and stop_pwm( ) ensure timers and PWM are disabled until the next transmission.

In some examples, the processing circuitry controlling these functions can be a STM32 microcontroller which has a timer generated PWM to an inductive coil. Receive circuitry coupled to the inductive coil can include one or more filters (e.g., a bandpass filter) and one or more amplifiers (e.g., a logarithmic amplifier) fed into a dynamically adjusted Vref comparator. The system has been tested to transmit and receive UART frames at standard baud rate or 2,400, but could be adjusted to work at other baud rates.

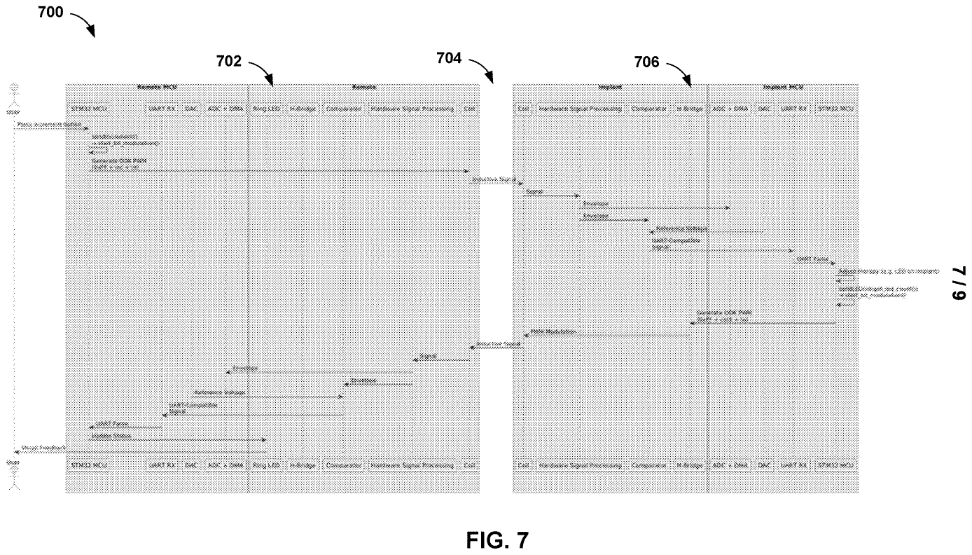

FIG. 7 is a sequence diagram 700 of an example sequence for transmitting and receiving information via inductive coupling. Steps within area 702 refer to transmitting steps, area 704 refers to the magnetic field generated from one inductive coil and received by another inductive coil, and area 706 refs to receiving steps in the process.

For example, a user can press an increment button (or other input) that is received by a microcontroller unit (MCU). The MCU can start bit modulation and generate OOK PWM signals for the inductive coil. The inductive coil generates a modulated magnetic field that is received by another inductive coil of the receiving device and sends a signal to hardware signal processing. The envelope is sent to the ADC and DMA and also to a comparator. The comparator receives a reference voltage from a DAC and generates a UART-compatible digital signal that is sent to the UART Rx. The UART Rx parses the signal. The MCU of the receiving device (such as of an IMD), can then control adjustment to therapy or other action.

The MCU of the receiving device can initiate transmission back to the transmitting device. For example, the MCU can start bit modulation and generate OOK PWM signals for the secondary inductive coil. The secondary inductive coil generates a modulated magnetic field that is received by the primary inductive coil of the transmitting device and sends a signal to hardware signal processing. Similar to the receiving device, the transmitting device can send the envelope to the ADC and DMA of the transmitting device and also to another comparator. The comparator receives a reference voltage from a DAC of the transmitting device and generates a UART-compatible digital signal that is sent to the UART Rx of the transmitting device. The UART Rx parses the signal. The MCU of the transmitting device (e.g., the external device), can then update the status of the IMD such as by controlling a ring LED to turn on, off, or change color which can be seen as visual feedback to the user.

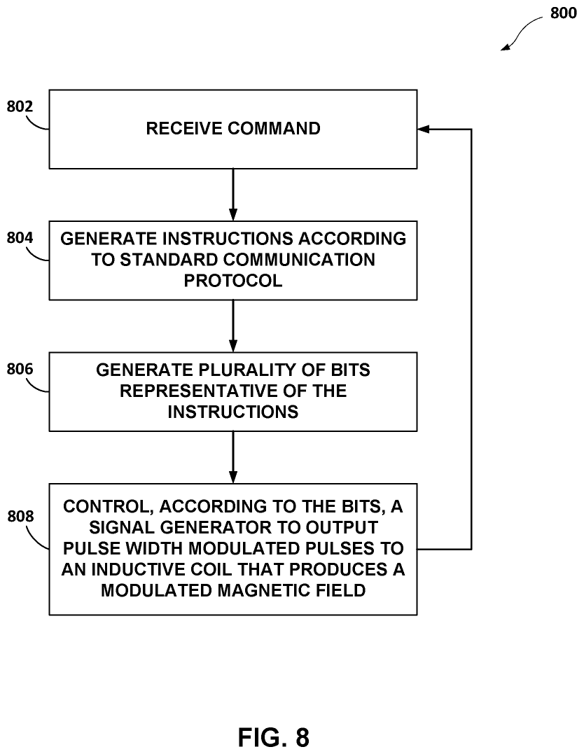

FIG. 8 is a flow chart illustrating an example technique 800 for transmitting information via inductive coupling. The process of technique 800 can be performed by any of the devices described herein, such as by IMDs 110, 310, or 410, or by any external devices 112, 312, or 550. For the purposes of describing transmission of a command from an external device to an IMD, external device 312 will be described in this example.

As shown in FIG. 8, processing circuitry 530 can receive a command, such as a request from a user via user interface 554 (802). Processing circuitry 530 can generate instructions according to a standard communication protocol (804) and then generate a plurality of bits representative of the instructions (806). Processing circuitry 530 can then control, according to the bits, a signal generator (e.g., inductive circuitry 568) to output pulse width modulated pulses to an inductive coil 528 that produces a modulated magnetic field according to the pulses (808). A receiving device can receive the modulated magnetic field via anther inductive coil.

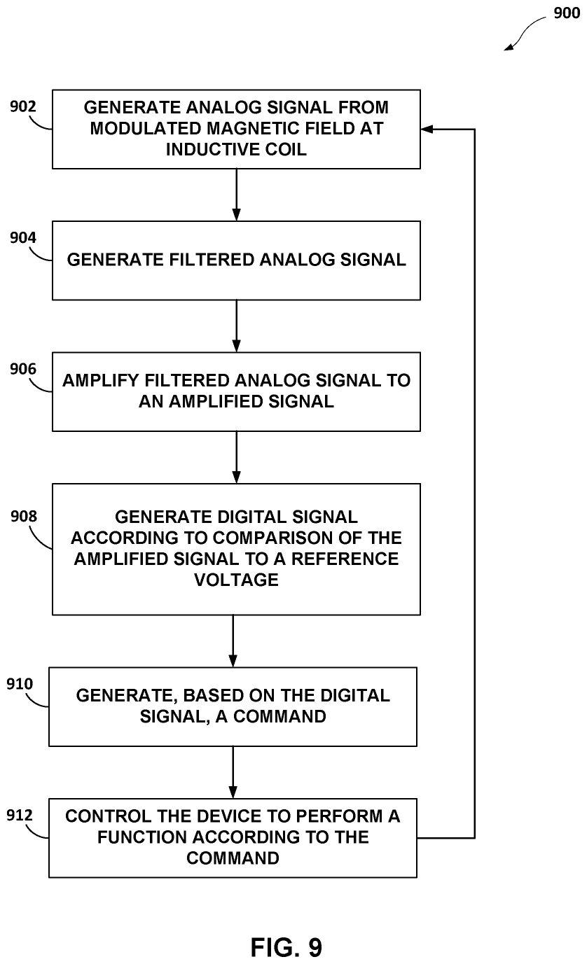

FIG. 9 is a flow chart illustrating an example technique 900 for receiving information and converting the information to digital information via inductive coupling. The process of technique 900 can be performed by any of the devices described herein, such as by IMDs 110, 310, or 410, or by any external devices 112, 312, or 550. For the purposes of describing receiving a command from an external device, IMD 410 will be described in this example.

As shown in FIG. 9, inductive circuitry 438 generates an analog signal from a modulated magnetic field detected at inductive coil 416 (902). A bandpass filter can generate a filtered analog signal from the analog signal (904), and an amplifier can amplify the filtered analog signal to an amplified signal (906). A comparator can generate a digital signal according to the comparison of the amplified signal to a reference voltage (908). Receive circuitry 450 of IMD 410 may include the bandpass filter, the amplifier, and the comparator. Processing circuitry 430 can then generate, based on the digital signal from the comparator, a command (910). The command may be in the format of the communication or interface protocol from the transmitting device, such as the UART format. Processing circuitry 430 can then control IMD 410 to perform a function according to the command (910). For example, the function may be to increase an amplitude of stimulation as instructed by the command.

In one or more examples, the functions described above may be implemented in hardware, software, firmware, or any combination thereof. For example, the various components of FIGS. 1-3, processing circuitry 430 and processing circuitry 530 may be implemented in hardware, software, firmware, or any combination thereof. If implemented in software, the functions may be stored on or transmitted over, as one or more instructions or code, a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that may be accessed by one or more computers or one or more processors to retrieve instructions, code and/or data structures for implementation of the techniques described in this disclosure. A computer program product may include a computer-readable medium.

The term “non-transitory” may indicate that the storage medium is not embodied in a carrier wave or a propagated signal. In certain examples, a non-transitory storage medium may store data that may, over time, change (e.g., in RAM or cache). By way of example, and not limitation, such computer-readable storage media, may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or other computer readable media. In some examples, an article of manufacture may include one or more computer-readable storage media.

Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transient media, but are instead directed to non-transient, tangible storage media. Combinations of the above should also be included within the scope of computer-readable media.

Instructions may be executed by one or more processors, such as one or more DSPs, general purpose microprocessors, ASICs, FPGAs, or other equivalent integrated or discrete logic circuitry. Accordingly, the term “processor” and “processing circuitry,” as used herein, may refer to any of the foregoing structure or any other structure suitable for implementation of the techniques described herein. Also, the techniques could be fully implemented in one or more circuits or logic elements.

The techniques of this disclosure may be implemented in a wide variety of devices or apparatuses, including, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperative hardware units, including one or more processors as described above, in conjunction with suitable software and/or firmware.

Various examples of the disclosure have been described. These and other examples are within the scope of the following claims.

What is claimed is:

1. A device comprising:
an inductive coil;
a signal generator coupled to the inductive coil; and
processing circuitry configured to:
receive a command;
responsive to receiving the command, generate instructions according to a standard communication protocol;
generate, based on the instructions, a plurality of bits representative of the instructions; and
control, according to the plurality of bits, the signal generator to output pulse width modulated pulses to the inductive coil that produces a modulated magnetic field.

2. The device of claim 1, wherein the processing circuitry is configured to generate the plurality of bits by at least determining a bit duration using a timer clock and a selected baud rate.

3. The device of claim 1, wherein the processing circuitry is configured to control the signal generator to output the pulse width modulate pulses by at least enabling or disabling pulse width modulation to represent each bit of the plurality of bits.

4. The device of claim 1, further comprising a user interface configured to receive a user input, and wherein the command comprises the user input.

5. The device of claim 1, wherein a resonant frequency of the inductive coil is from about 9 kHz to about 200 kHz.

6. The device of claim 1, wherein the processing circuitry comprises a microcontroller unit that includes a Universal Asynchronous Receiver-Transmitter (UART), and wherein the standard communication protocol comprises a UART protocol.

7. The device of claim 1, wherein the device comprises an external device.

8. The device of claim 7, wherein the external device comprises a mobile computing device.

9. The device of claim 7, wherein the external device comprises a fob comprising a single user interface having one or two physical buttons.

10. The device of claim 1, wherein the device comprises an implantable medical device.

11. A device comprising:
an inductive coil configured to generate an analog signal from a received modulated magnetic field;
a receive circuit configured to generate a digital signal from the analog signal; and
processing circuitry configured to:
generate, based on the digital signal, a command; and
control the device to perform a function according to the command.

12. The device of claim 11, wherein the receive circuit comprises:
a bandpass filter;
a logarithmic amplifier; and
a comparator.

13. The device of claim 12, wherein:
the bandpass filter is configured to generate a filtered analog signal from the analog signal;
the logarithmic amplifier is configured to amplify the filtered analog signal to an amplified signal; and
the comparator is configured to generate the digital signal according to a comparison of the amplified signal to a reference voltage.

14. The device of claim 11, wherein the digital signal is a reconstruction of instructions according to a standard communication protocol generated by a second device that generated the modulated magnetic field.

15. The device of claim 11, wherein the processing circuitry comprises a microcontroller unit that includes a Universal Asynchronous Receiver-Transmitter (UART), and wherein the command is in a standard communication protocol that comprises a UART protocol.

16. The device of claim 11, wherein a resonant frequency of the inductive coil is from about 9 kHz to about 200 kHz.

17. The device of claim 11, wherein the device comprises an external device.

18. The device of claim 17, wherein the external device comprises a mobile computing device or a fob comprising a single user interface having one or two physical buttons.

19. The device of claim 1, wherein the device comprises an implantable medical device.

20. A system comprising:
an external device comprising:
a first inductive coil;
a signal generator coupled to the first inductive coil; and
first processing circuitry configured to:
receive a first command;
responsive to receiving the first command, generate instructions according to a standard communication protocol;
generate, based on the instructions, a plurality of bits representative of the instructions; and
control, according to the plurality of bits, the signal generator to output pulse width modulated pulses to the first inductive coil that produces a modulated magnetic field; and
an implantable medical device comprising:
a second inductive coil configured to generate an analog signal from the modulated magnetic field;
a receive circuit comprising:
a bandpass filter configured to generate a filtered analog signal from the analog signal;
a logarithmic amplifier configured to amplify the filtered analog signal to an amplified signal; and
a comparator configured to generate a digital signal according to a comparison of the amplified signal to a reference voltage; and
second processing circuitry configured to:
generate, based on the digital signal, a second command; and
control the implantable medical device to perform a function according to the second command.
